(19) United States
(12) Patent Application Publication
(10) Pub. No.: US 2017/0342377 A1
(43) Pub. Date: Nov. 30, 2017

(54) CELL CULTURE CARRIER AND CELL CULTURE MODULE

(71) Applicant: COORSTEK KK, Tokyo (JP)

(72) Inventors: Takafumi IMAIZUMI, Kanagawa (JP); Shinya HASHIMOTO, Kanagawa (JP)

(21) Appl. No.: 15/607,239

(22) Filed: May 26, 2017

(30) Foreign Application Priority Data
May 27, 2016 (JP) 2016-106423
Apr. 7, 2017 (JP) 2017-076482

(51) Int. Cl. C12N 5/00; C12N 5/0735; C12N 5/0775; C12M 1/22

(52) U.S. Cl. CPC C12N 5/0068; C12N 5/0666; C12N 5/0606; C12N 5/0663; C12N 5/0668; C12N 5/0667; C12N 5/0665; C12N 5/0664; C12M 1/22; C12N 2533/14; C12N 2535/00

(57) ABSTRACT

A cell culture carrier includes a first substrate. The first substrate has a plurality of wells on an upper surface and includes a porous body. The porous body has pores with an average pore diameter of 0.05 μm or more and 10 μm or less, and has a porosity of 25% or more and 50% or less. The first substrate has a thickness from a lowermost portion of the well to a lower surface of the substrate of 50 μm or more and 10 mm or less.

### TECHNICAL FIELD

The present invention relates to a culture carrier and a culture module suitable for culturing undifferentiated cells such as embryonic stem cells (ES cells), induced pluripotent stem cells (iPS cells) and somatic stem cells.

### BACKGROUND ART

In recent years, as a result of the progress of separation of stem cells from human bone marrow fluid, differentiation or induction into target tissue cells, three-dimensional culture technique, development of scaffolding materials, and the like, it becomes possible to prepare tissues such as skin, bone, cartilage, blood vessel, heart valve, ligament, and the like from stem cells by cell culture, and in some cases, clinical application has already been started.

On the other hand, since undifferentiated cells represented by ES cells and iPS cells are totipotent cells capable of differentiating into all other tissue cells, they are cells expected to be applied to regenerative medicine. Therefore, studies on the method of inducing differentiation from the undifferentiated cells to tissue cells are actively progressed.

In order to use the ES cell and the iPS cell for the purpose of regenerative medicine, a technique for growing a large amount of these cells is required.

Conventionally, as a normal culture carrier for growing or maintaining the undifferentiated cells represented by the ES cells, the iPS cells and the like as described above, a culture carrier in which a plastic Petri dish is coated with an in vivo component such as collagen, gelatin, laminin, MATRIGEL, and the like and supporting cells derived from mouse or derived from human tissue are grown thereon is used.

For example, PTL 1 discloses a cell culture carrier in which a plurality of recessed portions (wells) of which surface is made of a porous body are arranged in a matrix form on a front surface of a substrate. FIG. 7 illustrates a schematic view of a structure of the cell culture carrier disclosed in PTL 1.

In the cell culture carrier 40, a plurality of recessed portions (wells) 41 of which surface is made of the porous body are arranged in a matrix form on the front surface of the substrate. By using the cell culture carrier 40 having such a shape, cells can be grown only in the recessed portions (wells) 41 formed on the front surface of the substrate, and size control of spheroids of undifferentiated cells such as ES cells can be performed.

In addition, the surface of the recessed portion 41 is configured with a porous body so that the cultured cells adequately adhere to the inside of the recessed portion 41 of the cell culture carrier 40 and are easily detached.

Therefore, when collecting the cultured cells, in particular, attachment and detachment of the cultured cells to and from the culture carrier can be performed more efficiently without requiring long-time enzymatic treatment that is undesirable in human iPS cells and the like,

### CITATION LIST

### Patent Literature

PTL 1: JP-2008-306987

### SUMMARY OF INVENTION

### Technical Problem

In the cell culture carrier disclosed in PTL 1, as described above, size control of spheroids of undifferentiated cells such as ES cells becomes possible, and attachment and detachment of the cultured cells to and from the culture carrier can be performed more efficiently.

However, as illustrated in FIG. 7, undifferentiated cells seeded on a front surface 40a of a cell culture carrier 40 are seeded on the front surface 40a of the cell culture carrier other than a plurality of recessed portions (wells) 41 by spontaneous sedimentation. This state is illustrated in FIG. 8. In FIG. 8, hatched portions represent regions seeded with the undifferentiated cells.

The cells seeded in this manner exist not only on the recessed portions (wells) 41 but also on the front surface 40a of the cell culture carrier other than the plurality of recessed portions (wells) 41.

Among the undifferentiated cells adhered to the front surface 40a of the cell culture carrier, cells that cannot form cell aggregation die, and cells forming the cell aggregation become non-uniform cell aggregation. Therefore, there was a technical problem that the undifferentiated cells such as ES cells, iPS cells and the like could not be efficiently cultured.

In order to solve the above technical problem, when seeding the undifferentiated cells such as ES cells, iPS cells and the like on the surface of the cell culture carrier, on the premise that the undifferentiated cells seeded on a region other than the plurality of recessed portions are guided into the recessed portions (wells) by suction from the rear surface side or/and pressurization from the front surface side of the cell culture carrier, the inventors of the application improved the cell culture carrier, and thus completed the present invention.

An object of the present invention is to provide a cell culture carrier and a cell culture module capable of forming spheroids (cell aggregation) having a uniform size by efficiently aggregating undifferentiated cells such as ES cells, iPS cells and the like, and growing cells while maintaining an undifferentiated state.

### Solution to Problem

To achieve the above object, a cell culture carrier according to the present invention includes a first substrate having a plurality of wells on an upper surface and including a porous body, the porous body having pores with an average pore diameter of 0.05 μm or more and 10 μm or less and having a porosity of 25% or more and 50% or less, in which the first substrate has a thickness from a lowermost portion of the well to a lower surface of the substrate of 50 μm or more and 10 mm or less.

Since the cell, culture carrier according to the present invention includes the first substrate including the above-described porous body having the specific pore diameter and the porosity, after the cells are seeded on the cell culture carrier, suction from the rear surface side or/and pressurization from the front surface side of the cell culture carrier can be performed.

In addition, since the thickness from the lowermost portion of the well of the first substrate to a lower surface of the substrate is configured to have 50 μm or more and 10 mm or less, when sucked from the rear surface side or/and pressurized from the front surface side of the cell culture carrier, a difference in pressure loss is generated between the front surface of the first substrate and the lowermost portion of the well.

The difference in pressure loss allows the cells on the front surface of the first substrate to be collected in the wells and the cells in the wells to be further collected to the lowermost portion of the well so that cells of uniform size can be conveniently cultured.

By applying pressure from the rear surface side of the cell culture carrier, the difference in pressure loss is generated between the front surface of the first substrate and the lowermost portion of the well, similarly to the case of suction, so that the cultured cell aggregation can be easily detached from the cell culture carrier. Therefore, cultured cells of uniform size can be conveniently obtained.

In this manner, by using the cell culture carrier according to the present invention, it is possible to uniformly collect cells to the lowermost portion of each well and to form a cell aggregation while maintaining an undifferentiated state. Therefore, it is possible to culture the cell aggregation having a uniform size in large quantity and efficiently while maintaining the undifferentiated state.

In addition, to achieve the above object, a cell culture carrier according to the present invention includes a first substrate having a plurality of wells on an upper surface, having a thickness from a lowermost portion of the well to a lower surface of the substrate of more than 0 μm and 45 μm or less, and including a porous body, the porous body having pores with an average pore diameter of 0.05 μm or more and 10 μm or less and having a porosity of 25% or more and 50% or less, and a second substrate having a communication hole with an average pore diameter of 50 μm or more and 200 μm or less and with an average pore diameter of a communication portion of 20 μm or more and 100 μm or less, and having a skeleton portion with an average pore diameter of 0.05 μm or more and 1.0 μm or less, in which the first substrate is stacked on an upper surface of the second substrate.

In this manner, the second substrate is provided on the rear surface (lower surface) of the first substrate having weak mechanical strength so that it is possible to efficiently suck cells into the well of the first substrate while preventing breakage of the first substrate.

Here, it is desirable that the well has a diameter of 10 μm or more and 1000 μm or less and a depth of 10 μm or more and 1000 μm or less.

Normally, a stem cell aggregation such as an ES cell aggregation or an iPS cell aggregation loses an undifferentiated state when reaching a certain size or more, and it is therefore preferable that the well has a size within the above range from the viewpoint of maintaining the undifferentiated state.

Furthermore, it is desirable that the first substrate and the second substrate independently include at least one kind of ceramic selected from the group consisting of zirconia, yttria, titanic, alumina, alumina-silica, silica, hydroxyapatite, and β-tricalcium phosphate.

These ceramics are preferably used because of the high biostability. It is preferable that the first substrate and the second substrate are constituted of the same material.

In addition, desirable is a cell culture module including the cell culture carrier and including a suction portion on a rear surface side or/and a pressurizing portion on a front surface side of the cell culture carrier.

### Advantageous Effects of Invention

According to the present invention, a cell culture carrier and a cell culture module capable of forming spheroids (cell aggregation) having a uniform size by efficiently aggregating undifferentiated cells such as ES cells, iPS cells and the like, and growing cells while maintaining an undifferentiated state can be obtained.

### BRIEF DESCRIPTION OF DRAWINGS

FIG. 1 is a conceptual diagram (perspective view) illustrating a first embodiment of a cell culture carrier of the present invention.

FIG. 2 is a cross-sectional view taken along line I-I of FIG. 1.

FIG. 3 is a conceptual diagram (perspective view) illustrating a second embodiment of the cell culture carrier of the present invention.

FIG. 4 is a cross-sectional view taken along fine II-II of FIG. 3.

FIG. 5 is a conceptual view (perspective view) of a second substrate of the second embodiment.

FIG. 6 is a schematic configuration diagram illustrating a cell culture module according to the present invention.

FIG. 7 is a conceptual view (perspective view) illustrating a conventional cell culture carrier.

FIG. 8 is a conceptual diagram (cross-sectional view) illustrating a state in which undifferentiated cells are seeded on the conventional cell culture carrier.

### DESCRIPTION OF EMBODIMENTS

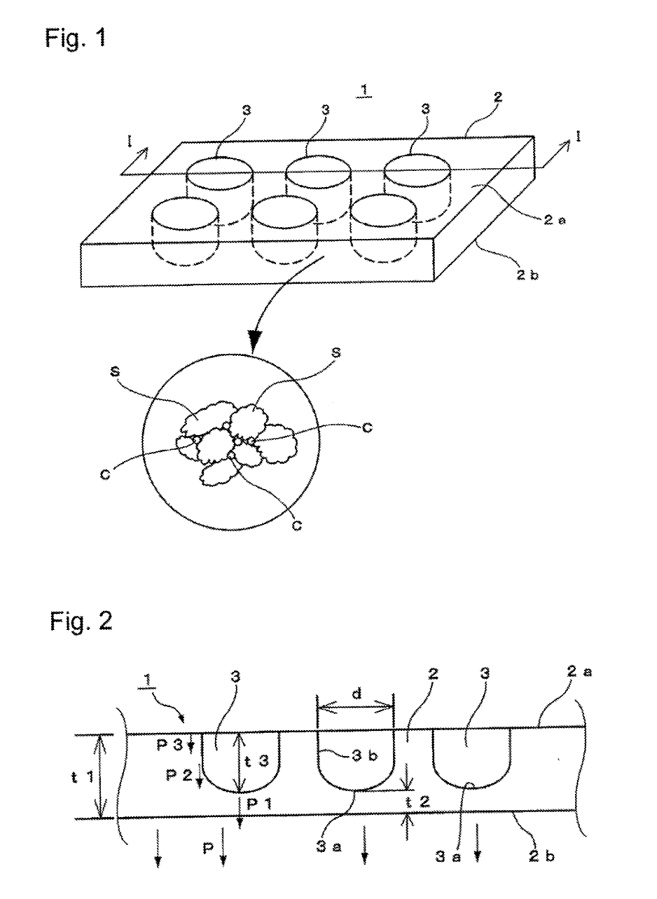

Hereinafter, a first embodiment of a cell culture carrier according to the present invention will be described with reference to FIG. 1 and FIG. 2.

A cell culture carrier 1 of the first embodiment is constituted of a substrate 2 (first substrate) having a plurality of wells 3 on an upper surface and made of a porous body, the porous body having pores c having an average pore diameter of 0.05 μm or more and 10 μm or less and having a porosity of 25% or more and 50% or less, The substrate 2 (first substrate) is formed of a ceramic porous sintered body. As illustrated in. FIG. 1, fme pores c having a specific diameter are formed between ceramic particles S and are formed to have a specific porosity.

In addition, the thickness t2 from the lowermost portion 3a of the well of the substrate 2 (first substrate) to the lower surface 2b of the substrate is formed to be 50 μm or more and 10 mm or less.

In this manner, since the cell culture carrier 1 is constituted of the porous body having a specific pore diameter and porosity in the substrate 2 (first substrate), when undifferentiated cells are seeded on the cell culture carrier 1, suction from the rear surface side of the cell culture carrier 1 (substrate 2) can be performed.

In addition, since the thickness t2 from the lowermost portion 3a of the well of the first substrate 2 to the lower surface 2b of the substrate is configured to have 50 μm or more and 10 mm or less, when sucked from the rear surface side of the cell culture carrier 1 (substrate 2), a difference in pressure loss is generated between the front surface 2a of the substrate 2 and the lowermost portion 3a of the well 3.

That is, a large negative pressure is generated at the lowermost portion 3a of the well, and a small negative pressure is generated (or no negative pressure is generated) on the front surface 2a of the substrate, and therefore the undifferentiated cells on the front surface 2a of the substrate can be collected in the well and the undifferentiated cells in the well 3 can be further collected to the lowermost portion (lowermost portion 3a of well) of the well 3.

More preferably, the thickness t2 from the lowermost portion 3a of the well of the substrate 2 (first substrate) to the lower surface 2b of the substrate is desirably formed to be 50 μm or more and 500 μm or less,

Here, the substrate 2 (first substrate) made of the porous body having the pores having the average pore diameter of 0.05 μm or more and 10 μm or less and having the porosity of 25% or more and 50% or less is used in order to generate a larger pressure loss difference with a small length dimension in the thickness direction of the substrate 2.

In addition, by setting the specific average pore diameter as described above, the cultured cells can adequately adhere to the well 3 of the cell culture carrier 1 and can be detached more easily.

Specifically, when the average pore diameter exceeds 10 μm, the pressure loss is low and it is not possible to uniformly suck the cells, which is not preferable, and in a case where it is less than 0.05 μm, the pressure loss becomes large, which is not preferable.

In addition, when the porosity exceeds 50%, the strength of the substrate 2 is low, which is not preferable, and in a case where it is less than 25%, the pressure loss increases, which is not preferable.

More preferably, the average pore diameter is desirably formed to be 0.05 μm or more and 5 μm or less, and the porosity to be 30% or more and 50% or less.

The diameter d of the well is formed to be 10 μm or more and 1000 μm or less, and the depth t3 to be 10 μm or more and 1000 μm or less.

Normally, the ES cell aggregation and the iPS cell aggregation loses the undifferentiated state when reaching a certain size or more, and it is therefore preferable that the well 3 has a size within the above range from the viewpoint of maintaining the undifferentiated state.

More preferably, the diameter d of the well is desirably formed to be 50 μm or more and 500 μm or less, and the depth t3 to be 50 μm or more and 500 μm or less.

In addition, the size of the well 3 is preferably a diameter of 2 times or more and 50 times or less the size of the cells to be cultured.

The cell aggregation grown in the well 3 of the size as described above can be made uniform in size and are easy to recover by pipetting or the like so that an efficient culture can be performed.

A well 3 having a size one times the size of the cell can maintain the undifferentiated state, but there is not much room for growth and it is preferable that the cells do not protrude from the well 3. Therefore, it is more preferable that the well 3 has a diameter 5 times or more the size of the cells to be cultured. In addition, it is more preferably 25 times or less in order to enhance the certainty of undifferentiating.

The shape of the well 3 is not particularly limited, and various shapes can be adopted as long as the front surface 2a of the substrate 2 can be processed in the above size.

Specifically, in a ease that the cell aggregation detached from the cell culture carrier 1 according to the present invention is used for suspension culture, it is preferable that the bottom surface has a hemispherical shape from the viewpoint of detachability, the shape of the aggregation to be formed, and the like.

In the present invention, the size of the well 3 is expressed by the diameter and the depth. In a case where the opening surface is a polygonal shape, the diameter d of the well 3 referred to in the present invention is a diameter in a case where the opening area of the well 3 on the front surface 2a of the substrate 2 of the cell culture carrier 1 is replaced by that of a circle. In addition, the depth t3 is a depth of the deepest portion of the well 3.

As the material of the cell culture carrier 1 according to the present invention, a nonmetallic inorganic material is preferable, and ceramic is particularly preferably used. Examples of specific materials include zirconia, yttria, titania, alumina, silica, alumina-silica, hydroxyapatite, β-tricalcium phosphate, and the like with high biological safety, and among these, alumina, zirconia, hydroxyapatite, β-tricalcium phosphate, and titania are more preferable, in which biostability is confirmed. Specifically, zirconia or alumina is preferable.

The cell culture carrier 1 is a porous sintered body prepared by using the above material, thereby it is possible to sufficiently supply the culture liquid to the cells adhered to the culture carrier.

The thickness t2 from the lowermost portion 3a of the well to the lower surface (rear surface) 2b of the substrate 2 is formed to be 50 μm or more and 10 μm or less.

In a case where the thickness t2 from the lowermost portion 3a of the well to the lower surface (rear surface) 2b of the substrate 2 is large, the pressure loss is large and thus a large negative pressure cannot be applied to the lowermost portion 3a of the well, and in a case where the thickness t2 from the lowermost portion 3a of the well to the lower surface 2b of the substrate 2 is small, the mechanical strength of the substrate 2 is weak, which are not preferable.

More preferably, the thickness t2 from the lowermost portion 3a of the well to the lower surface (rear surface) 2b of the substrate 2 is desirably formed to be 50 μm or more and 500 μm or less.

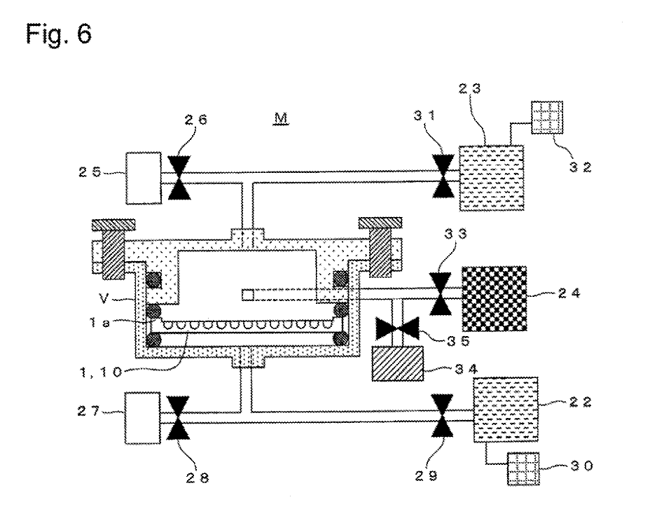

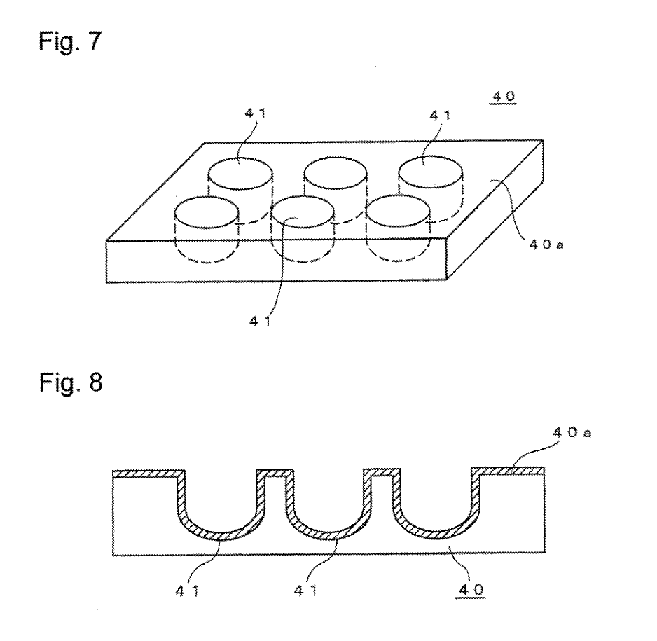

The shape of the substrate (for example, a carrier as illustrated in FIG. 1, or a so-called banked carrier having a ring-shaped stepped portion la in the outer peripheral portion as illustrated in FIG. 6) of the first substrate is not particularly limited as long as the thickness t2 front the lowermost portion 3a of the well to the lower surface (rear surface) 2b of the substrate 2 is in the above range, but the thickness t1 of the substrate (first substrate) is preferably 0.1 mm or more and 12 mm or less, and more preferably 0.1 mm or more and 5.0 mm or less.

As illustrated in FIG. 2, when suction is performed with the pressure P from the rear surface side of the cell culture carrier 1 configured in this manner, a negative pressure P1 acts on the lowermost portion 3a of the well, a negative pressure P2 smaller than the negative pressure P1 acts on the side wall portion 3b of the well, and a negative pressure P3 smaller than the negative pressure P2 further acts on the front surface 2a of the cell culture carrier 1 (substrate 2).

Due to the negative pressure difference generated by the difference in pressure loss, the cells on the front surface 2a of the substrate can be collected in the well 3, and the cells in the well 3 can be further collected to the central portion of the bottom surface portion of the well 3 (the lowermost portion 3a of the well 3), so that the cells of uniform size can be easily cultured.

By using the cell culture carrier 1 according to the present invention, it is possible to uniformly collect the undifferentiated cells to the bottom surface portion (the lowermost portion 3a) of each well 3 and to form the cell aggregation while maintaining an undifferentiated state. Therefore, it is possible to culture the cell aggregation having a uniform size in large quantity and efficiently while maintaining the undifferentiated state.

By applying a positive pressure (pressurizing) from the rear surface side of the cell culture carrier 1 (substrate 2), the difference in pressure loss is generated between the front surface 2a of the substrate 2 and the lowermost portion 3a of the well in the same manner as in the case of suction, so that the cultured cell aggregation can also be easily detached from the cell culture carrier 1.

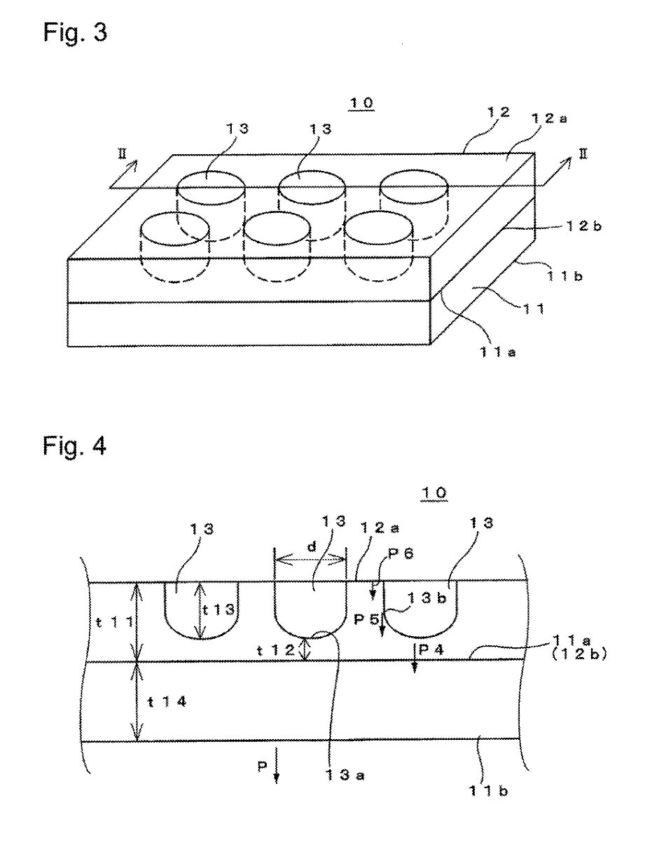

Next, a second embodiment of a cell culture carrier according to the present invention will be described with reference to FIG. 3 and FIG. 4.

In the second embodiment, a second substrate 11 is stacked on a rear surface 12b of a substrate (first substrate) 12.

Specifically, as in the first substrate 2 of the first embodiment, in a case where the substrate (first substrate) 12 has, for example, a plurality of wells on the upper surface and is formed of a porous body having pores having an average pore diameter of 0.05 μm or more and 10 μm or less and having a porosity of 25% or more and 50% or less, in which the thickness from the lowermost portion of the well to the lower surface of the substrate is formed to be more than 0 μm and 45 μm or less, and the mechanical strength is weak, the second substrate 11 is stacked.

In addition to increasing the mechanical strength of the first substrate 12, in order to more uniformly suck the rear surface 12b of the first substrate 12 when sucking from the rear surface of the second substrate 11, the second substrate 11 is disposed on a rear surface (lower surface) 12b of the first substrate 12.

Similar to the first substrate 2, the second substrate 11 is constituted of at least one kind of ceramic selected from the group consisting of zirconia, yttria, titania, alumina, silica, alumina-silica, hydroxyapatite, and β-tricalcium phosphate. As described above, the first substrate 12 is stacked on the upper surface of the second substrate 11.

The first substrate 12 and the second substrate 11 are ceramic porous sintered bodies and are preferably constituted of the same material. In this manner, the substrate (first substrate) 12 and the second substrate 11 can be efficiently and firmly sintered.

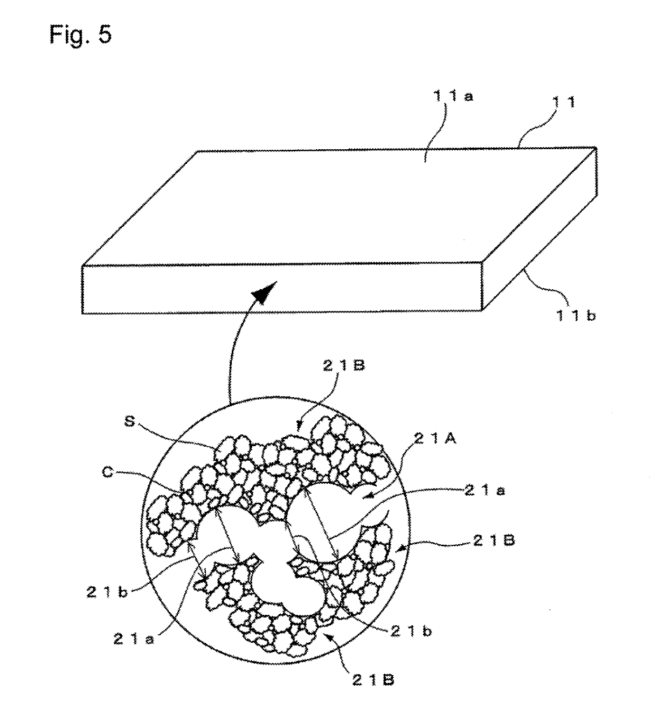

As illustrated in FIG. 5, the second substrate 11 has a communication hole 21A having an average pore diameter of a pore portion 21a of 50 μm or more and 200 μm or less and an average pore diameter of a communication portion 21b of 20 μm or more and 100 μm or less, and a skeleton portion 21B having an average pore diameter of 0.05 μm or more and 1.0 μm or less.

That is, the second substrate 11 has a three-dimensional mesh-like skeleton structure in which adjacent portions of multiple of spherical pore portions 21a communicate with each other via the communication portion 21b.

Specifically, the average pore diameter of the pore portion 21a measured by using a mercury porosimeter (based on JIS R 1634 1998) is 50 μm or more and 200 μm or less, and the three-dimensional mesh-like skeleton structure is provided in which the pore portions 21a communicate with each other through the communication portions 21b of 20 μm or more and 100 μm or less (based on SEM observation).

Here, in a case where the average pore diameter of the pore portion 21a is less than 50 μm, the pressure loss increases and the suction or pressurizing effect cannot be obtained, which is not preferable. In addition, in a case where the average pore diameter of the pore portion 21a exceeds 200 μm, the mechanical strength of the second substrate 11 becomes weak and a reinforcing effect cannot be sufficiently obtained, which is not preferable.

hi addition, in a case where the average pore diameter of the communication portion 21b is less than 20 μm, the pressure loss increases and the suction and pressurizing effect cannot be obtained, which are not preferable. In addition, in a case where the average pore diameter of the communication portion 21b exceeds 100 μm, the mechanical strength of the second substrate 11 becomes weak and a reinforcing effect cannot be sufficiently obtained, which is not preferable.

It is preferable that the average pore diameter of pores c in the skeleton portion 21B is 0.05 μm or more and 1.0 μm or less.

In a case where the average pore diameter of pores c in the skeleton portion 21B is less than 0.05 μm, the strength of the second substrate 11 becomes large and the processing efficiency deteriorates when processing to a desired shape. In a case where it exceeds 1 μm, a raw material having larger particles is required, and it is difficult to prepare the second substrate.

Furthermore, it is preferable that the porosity (calculated from the volume, weight, and density by machining the sample into a specific volume and measuring the weight) of the second substrate 11 is formed to be 60% or more and 85% or less. When the porosity of the second substrate 11 is 60% or more and 85% or less, it is possible to have a reinthrcing effect on the first substrate without impairing the suction or pressurizing effect.

As illustrated in FIG. 3 and FIG. 4, it is preferable that the second substrate 11 formed in this manner has a thickness t14 of 1 mm or more and 5 mm or less. When the thickness t is set to be 1 mm or more and 5 mm or less, it is possible to have a reinforcing effect on the first substrate without impairing the suction or pressurizing effect.

By stacking the first substrate 12 on the upper surface (front surface) 11a of the second substrate 11, a cell culture carrier 10 is constituted. The stacking may be only overlapping the first substrate and the second substrate, or may be integrating by adhesion or the like. In a case of integrating by adhesion or the like, it can be performed by a known method, but, for example, it can be obtained by applying the same raw material particles as the first substrate to the rear surface 12b of the first substrate 12, stacking on the upper surface 11a of the second substrate, and pressure sintering.

As illustrated in FIG. 4, when suction is performed with the pressure P from the rear surface side (rear surface 11b side of the second substrate 11) of the cell culture carrier 10 configured in this manner, a negative pressure P4 acts on the lowermost portion 13a of the well, a negative pressure P5 smaller than the negative pressure P4 acts on the side wall portion 13b of the well, and a negative pressure P6 smaller than the negative pressure P5 further acts on the front surface (front surface 12a of first substrate 12) of the cell culture carrier 10.

Due to the negative pressure difference generated by the difference in pressure loss, the cells on the front surface 12a of the first substrate 12 can be collected in the well 13, and the cells in the well 13 can be further collected to the bottom surface portion of the well 13 (the lowermost portion 13a of the well 13), so that the cells of uniform size can be easily cultured.

By using the cell culture carrier 10 according to the second embodiment, it is possible to uniformly collect the cells to each of the bottom surface portion of well 13 (the lowermost portion 13a of well 13) and to form the cell aggregation while maintaining an undifferentiated state. Therefore, it is possible to culture the cell aggregation having a uniform size in large quantity and efficiently while maintaining the undifferentiated state.

By applying a positive pressure (pressurizing) from the rear surface side (rear surface side 11b of second substrate 11) of the cell culture carrier 10 in the same manner as in the first embodiment, the difference in pressure toss is generated between the front surface 12a of the first substrate 12 and the lowermost portion 13a of the well similarly to the case of suction, so that the cultured cell aggregation can be easily detached from the cell culture carrier.

Next, a cell culture module according to the present invention will be described with reference to FIG. 6.

As illustrated in FIG. 6, a cell culture module M is provided with a container main body V for holding and accommodating the cell culture carrier 1, 10, a first culture liquid water pressure supply unit 22 for supplying the culture liquid into the container main body V, a second culture liquid water pressure supply unit 23 for supplying culture liquid into the container main body V, and a cell collection unit 24 that is open to the atmosphere for collecting the cultured cells.

In addition, the cell culture module M is provided with a pressurizing pump 25 (pressurizing unit) and a pressurizing control unit 26 for pressurizing the front surface side of the cell culture carrier 1, 10, and a vacuum pump 27 (suction unit) and a pressure control unit 28 for depressurizing the rear surface side of the cell culture carrier 1, 10.

Furthermore, the cell culture module M is provided with a first liquid amount control unit 29 and a first water pressure (liquid pressure) control unit 30 which supply the culture liquid into the container V, a second liquid amount control unit 31 and a second water pressure (liquid pressure) control unit 32 fore supplying the culture liquid into the container V, and a discharge amount control unit 33 for controlling the discharge amount of the culture liquid from the container V to the cell collection unit 24.

In addition, a culture liquid discharge unit 34 that is open to the atmosphere for discharging the culture liquid from the container V, and a culture liquid discharge amount control unit 35 for controlling the discharge amount of the culture liquid are provided.

In such a cell culture method using the cell culture module M, the cell culture carrier 1, 10 is accommodated in the container V and then, for example, a suspension containing iPS cells is seeded on the cell culture carrier 1, 10, followed by sucking from the rear surface side of the cell culture carrier by using the vacuum pump 27. Alternatively, after seeding, pressurization is performed by using the pressurizing pump from the front surface side of the cell culture carrier 1, 10.

In this manner, after seeding, by suctioning from the rear surface side or/and pressurizing from the front surface side of the cell culture carrier 1, 10, a difference in pressure loss is generated in the cell culture carrier 1, 10. Due to the pressure difference, the cells on the front surface of the cell culture carrier 1, 10 can be collected in the well, and the cells in the well can be further collected to the lowermost portion 3a of the well.

Next, the pressurizing control unit 26 is set to the “closed” state, the pressure control unit 26 is set to the “closed” state, the second liquid amount control unit 31 of the second culture liquid water pressure supply unit 23 is set to the “closed” state, the discharge amount control unit 33 of the cell collection unit 24 is set to the “closed” state, the first liquid amount control unit 29 of the first culture liquid water pressure supply unit 22 is set to the “open” state, and the culture liquid discharge amount control unit 35 is set to the “open” state.

Furthermore, supply pressure is applied from the first water pressure (liquid pressure) control unit 30, and the culture liquid is supplied until the container V is filled with the culture liquid. Here, in the second culture liquid water pressure supply unit 23, the culture liquid may be supplied by controlling the second liquid amount control unit 31 and the second water pressure (liquid pressure) control unit 32 in the same manner. Alternatively, the culture liquid may be supplied only by the second culture liquid water pressure supply unit 23.

When the container V is filled with the culture liquid, the supply pressure from the first culture liquid water pressure supply unit 22 is stopped, the first liquid amount control unit 29 is set to the “closed” state, and thereby the supply of the culture liquid in the first water pressure (liquid pressure) control unit 30 is stopped. Here, the culture liquid discharge amount control unit 35 is set to the “closed” state in the same manner,

Next, the second liquid amount control unit 31 of the second culture liquid water pressure supply unit 23 is set to the “open” state, and the supply pressure from the second water pressure (liquid pressure) control unit 32 is adjusted so that the cells are continuously cultured while supplying the culture liquid to the front surface side of the cell culture carrier 1, 10 at a constant rate. Here, the culture liquid discharge amount control unit 35 of the culture liquid discharge unit 34 opened to the atmosphere is set to the “open” state, and a new culture liquid is supplied to the cells by discharging the culture liquid.

After the cell culture, the supply pressure from the second water pressure (liquid pressure) control unit 32 is stopped, the second liquid amount control unit 31 is set to the “closed” state, and thereby the supply of the culture liquid is stopped, and the culture liquid discharge amount control unit 35 is also set to the “closed” state.

Thereafter, the discharge amount control unit 33 of the cell collection unit 24 is set to the “open” state to start discharging the culture liquid in the container V, and the first liquid amount control unit 29 of the first culture liquid water pressure supply unit 22 is set to the “open” state and the supply pressure is applied from the first water pressure (liquid pressure) control unit 30 to newly start to supply the culture liquid into the container V.

This operation causes liquid pressure to be supplied from the first culture liquid water pressure supply unit 22 to the rear surface side of the cell culture carrier 1, 10. This liquid pressure causes the cells cultured in the well to be detached from the front surface of the well, and the detached cells float in the culture liquid in the container V and are collected as it is in the cell collection unit 24 with the culture liquid.

As described above, in the cell culture module according to the present invention, a suction portion is provided on the rear surface side or/and a pressurizing portion is provided on the front surface side of the cell culture carrier in cell seeding and therefore, a difference in pressure loss is generated between the front surface side and the rear surface side of the cell culture carrier, and due to the pressure difference, the cells on the front surface of the cell culture carrier can be collected in the well.

In addition, since the supply pressure is applied to the culture liquid to culture the cells, cells with high resistance to pressure can be cultured. Furthermore, after culturing, cultured cells can be detached from the cell culture carrier by adding liquid force to the rear surface side of the cell culture carrier, and therefore there is an advantage that it is not required to perform enzyme treatment such as trypsin treatment or the like.

### EXAMPLES

Hereinafter, the present invention will be described more specifically based on Examples, but the present invention is not limitedly interpreted by the following Examples.

A first substrate was prepared by the following producing method.

### <Preparation of First Substrate and Evaluation of Cell Culture Carrier>

### Example 1

200 g of alumina powder (AKP-20, manufactured by Sumitomo Chemical Co., Ltd.) having an average particle diameter of 500 μm as a raw material, 4 g of polyacrylic acid ammonium (ARON A-30SL, manufactured by Toagosei Co., Ltd.) and 7 g of an epoxy resin (DENACOL EX-614B, manufactured by Nagase ChemteX Corporation) as dispersing agents, and 50 g of pure water as a dispersion solvent were stirred and mixed for 15 hours by a ball mill to prepare a raw material slurry.

Furthermore, 2 g of 3,3′-diaminodipropylamine (manufactured by Tokyo Chemical Industry Co., Ltd.) as a gelling agent was added, followed by pouring into a mold to obtain an alumina molded product. The molded product was sintered at 1100° C. for 2 hours to obtain an alumina ceramic sintered body. Multiple prescribed wells were formed in the obtained sintered body to provide a first substrate.

The thickness of the substrate was set to 0.5 mm, and the thickness from the lowermost portion of the well to the lower surface of the substrate was set to 200 μm. In addition, the diameter of the above well was set to 300 μm and the depth was set to 300 μm. The average pore diameter of the substrate was 0.18 μm, and the porosity was 40%.

On this substrate was seeded a suspension (5×10⁵ cells/ml) in which 5×10⁵ human iPS cells were suspended in 1 ml of PBS (phosphate buffered saline), followed by sucking at −99 kPa from the bottom of the substrate by using a vacuum pump. Thereafter, ALP (alkaline phosphatase) staining was performed, and the adhesion status of the cells on the upper surface and the recessed portions of the substrate was observed with a digital microscope. As a result, as shown in Table 1, it was confirmed that 90% of the seeded cells were adhered to inside the wells of the carrier.

### Example 2

180 g of alumina powder (TM-DAR, manufactured by Taimei Chemical Co., Ltd.) having an average particle diameter of 100 nm as a raw material, 4 g of polyacrylic acid ammonium (ARON A-30SL, manufactured by Toagosei Co., Ltd.) and 7 g of an epoxy resin (DENACOL EX-614B, manufactured by Nagase ChemteX Corporation) as dispersing agents, and 50 g of pure water as a dispersion solvent were stirred and mixed for 15 hours by a ball mill to prepare a raw material slurry.

Furthermore, 2 g of 3,3′-diaminodipropylamine (manufactured by Tokyo Chemical Industry Co., Ltd.) as a gelling agent was added, followed by pouring into a mold to obtain an alumina molded product.

The molded product was sintered at 1200° C. for 2 hours to obtain an alumina ceramic sintered body. Multiple prescribed wells were formed in the obtained sintered body to provide a first substrate.

The thickness of the substrate was set to 0.35 mm, and the thickness from the lowermost portion of the well to the lower surface of the substrate was set to 50 μm. In addition, the diameter of the above well was set to 300 μm and the depth was set to 300 μm. The average pore diameter of the substrate was 0.05 μm, and the porosity was 30%.

On this substrate was seeded a suspension (5×10⁵ cells/ml) in which 5×10⁵ human iPS cells were suspended in 1 ml of PBS (phosphate buffered saline) in the same manner as in Example 1, followed by sucking at −99 kPa from the bottom of the substrate by using a vacuum pump. Thereafter, ALP (alkaline phosphatase) staining was performed, and the adhesion status of the cells on the upper surface and the recessed portions of the substrate was observed with a digital microscope. As a result, as shown in Table 1, it was confirmed that 75% of the seeded cells were adhered to inside the wells of the carrier.

### Comparative Example 1

180 g of alumina powder (TM-DAR, manufactured by Taimei Chemical Co., Ltd.) having an average particle diameter of 100 nm as a raw material, 4 g of polyacrylic acid ammonium (ARON A-30SL, manufactured by Toagosei Co., Ltd.) and 7 g of an epoxy resin (DENACOL EX-614B, manufactured by Nagase ChemteX Corporation) as dispersing agents, and 50 g of pure water as a dispersion solvent were stirred and mixed for 15 hours by a ball mill to prepare a raw material slurry.

Furthermore, 2 g of 3,3′-diaminodipropylamine (manufactured by Tokyo Chemical Industry Co., Ltd.) as a gelling agent was added, followed by pouring into a mold to obtain an alumina molded product.

The molded product was sintered at 1300° C. for 2 hours to obtain an alumina ceramic sintered body. Multiple prescribed wells were formed in the obtained sintered body to provide a first substrate.

The thickness of the substrate was set to 0.5 mm, and the thickness from the lowermost portion of the well to the lower surface of the substrate was set to 200 μm. In addition, the diameter of the above well was set to 300 μm and the depth was set to 300 μm.

The average pore diameter of the substrate was 0.04 μm, and the porosity was 30%.

On this substrate was seeded a suspension (5×10⁵ cells/ml) in which 5×10⁵ human iPS cells were suspended in 1 ml of PBS (phosphate buffered saline) in the same manner as in Example 1, followed by sucking at −99 kPa from the bottom of the substrate by using a vacuum pump. Thereafter, ALP (alkaline phosphatase) staining was performed, and the adhesion status of the cells on the upper surface and the recessed portions of the substrate was observed with a digital microscope. As a result, as shown in Table 1, only 50% of the seeded cells were adhered to inside the wells of the carrier. It is considered that this was because the pressure loss increased and thus sucking was not sufficient.

Substrates of Examples 3 to 5 and Comparative Examples 2 to 4 as shown in Table 1 were prepared in the same process as in Example 1 except that the pore size and the porosity were changed depending on the firing temperature and the firing time. Thereafter, a suspension (5×10⁵ cells/ml) in which 5×10⁵ human iPS cells were suspended in 1 ml of PBS (phosphate buffered saline) was seeded thereon, followed by sucking at −99 kPa from the bottom of the substrate by using a vacuum pump. Thereafter, ALP (alkaline phosphatase) staining was performed, and the adhesion status of the cells on the upper surface and the recessed portions of the substrate was observed with a digital microscope. The adhesion status of the cells in the wells, which is the result, is shown in Table 1. In Comparative Examples 3 and 4, mechanical strength was small and the substrates were cracked.

### Example 6

200 g of fused alumina fine powder (LA 800 manufactured by Pacific Rundum. Co., Ltd.) as a raw material, 4 g of polyacrylic acid ammonium (ARON A-30SL, manufactured by Toagosei Co., Ltd.) and 7 g of an epoxy resin (DENACOL EX-614B, manufactured by Nagase ChemteX Corporation) as dispersing agents, and 50 g of pure water as a dispersion solvent were stirred and mixed for 15 hours by a ball mill to prepare a raw material slurry. Furthermore, 2 g of 3,3′-diaminodipropylamine (manufactured by Tokyo Chemical Industry Co., Ltd.) as a gelling agent was added, followed by pouring into a mold to obtain an alumina molded product. The molded product was sintered at 1800° C. in air for 2 hours to obtain an alumina ceramic sintered body.

Multiple prescribed wells were formed in the obtained sintered body to provide a first substrate. The thickness of the substrate was set to 2.3 mm, and the thickness from the lowermost portion of the well to the lower surface of the substrate was set to 2 mm. In addition, the diameter of the above well was set to 300 μm and the depth was set to 300 μm.

The average pore diameter of the substrate was 10 μm, and the porosity was 30%. On this substrate was seeded a suspension (5×10 cells/ml) in which 5×10⁵ human iPS cells were suspended in 1 ml of PBS (phosphate buffered saline), followed by sucking at 10 kPa from the bottom of the substrate by using a vacuum pump. Thereafter, ALP (alkaline phosphatase) staining was performed, and the adhesion status of the cells on the upper surface and the recessed portions of the substrate was observed with a digital microscope.

As a result, as shown in Table 1, it was confirmed that 60% of the seeded cells were adhered to inside the wells of the carrier.

### Example 7

200 g of alumina powder (AS-50, manufactured by Showa Denko K.K.) having an average particle diameter of 9 μm as a raw material, 1 g of polyacrylic acid ammonium (ARON A-30SL, manufactured by Toagosei Co., Ltd.) and 3.5 g of an epoxy resin (DENACOL EX-614B, manufactured by Nagase ChemteX Corporation) as dispersing agents, and 50 g of pure water as a dispersion solvent were stirred and mixed for 15 hours by a ball mill to prepare a raw material slurry. Furthermore, 1 g of 3,3′-diaminodipropylamine (manufactured by Tokyo Chemical Industry Co., Ltd.) as a gelling agent was added, followed by pouring into a mold to obtain an alumina molded product. The molded product was sintered at 1800° C. in air for 2 hours to obtain an alumina ceramic sintered body.

Multiple prescribed wells were formed in the obtained sintered body to provide a first substrate. The thickness of the substrate was set to 2.3 mm, and the thickness from the lowermost portion of the well to the lower surface of the substrate was set to 2 mm. In addition, the diameter of the above well was set to 300 μm and the depth was set to 300 μm.

The average pore diameter of the substrate was 2 μm, and the porosity was 25%. On the substrate was seeded a suspension (5×10⁵ cells/ml) in which 5×10⁵ human iPS cells were suspended in 1 ml of PBS (phosphate buffered saline), followed by sucking at −10 kPa from the bottom of the substrate by using a vacuum pump. Thereafter, ALP (alkaline phosphatase) staining was performed, and the adhesion status of the cells on the upper surface and the recessed portions of the substrate was observed with a digital microscope.

As a result, as shown in Table 1, it was confirmed that 60% of the seeded cells were adhered to inside the wells of the carrier.

### Example 8

200 g of alumina powder (AS-50, manufactured by Showa Denko K.K.) having an average particle diameter of 9 μm as a raw material, 1 g of polyacrylic acid ammonium (ARON A-30SL, manufactured by Toagosei Co., Ltd.) and 3.5 g of an epoxy resin (DENACOL EX-614B, manufactured by Nagase ChemteX Corporation) as dispersing agents, and 50 g of pare water as a dispersion solvent were stirred and mixed for 15 hours by a ball mill to prepare a raw material slurry. Furthermore, 1 g of 3,3′-diaminodipropylamine (manufactured by Tokyo Chemical Industry Co., Ltd.) as a gelling agent was added, followed by pouring into a mold to obtain an alumina molded product. The molded product was sintered at 1700° C. in air for 2 hours to obtain an alumina ceramic sintered body.

Multiple prescribed wells were formed in the obtained sintered body to provide a first substrate. The thickness of the substrate was set to 10.3 mm, and the thickness from the lowermost portion of the well to the lower surface of the substrate was set to 10 mm. In addition, the diameter of the above well was set to 300 μm and the depth was set to 300 μm.

The average pore diameter of the substrate was 2 μm, and the porosity was 35%. On this substrate was seeded a suspension (5×10⁵ cells mi) in which 5×10⁵ human iPS cells were suspended in 1 ml of PBS (phosphate buffered saline), followed by sucking at −10 kPa from the bottom of the substrate by using a vacuum pump. Thereafter, ALP (alkaline phosphatase) staining was performed, and the adhesion status of the cells on the upper surface and the recessed portions of the substrate was observed with a digital microscope.

As a result, as shown in Table 1, it was confirmed that 60% of the seeded cells were adhered to inside the wells of the carrier.

### Comparative Example 5

An alumina ceramic sintered body was prepared under the same conditions as in Example 6, except that the air atmosphere of the firing condition in Example 6 was changed to a hydrogen atmosphere.

Multiple prescribed wells were formed in the obtained sintered body to provide a first substrate. The thickness of the substrate was set to 2.3 mm, and the thickness from the lowermost portion of the well to the lower surface of the substrate was set to 2 mm. In addition, the diameter of the above well was set to 300 μm and the depth was set to 300 μm.

The average pore diameter of the substrate was 11 μm, and the porosity was 40%, On this substrate was seeded a suspension (5×10⁵ cells/ml) in which 5×10⁵ human iPS cells were suspended in 1 ml of PBS (phosphate buffered saline), followed by sucking at −10 kPa from the bottom of the substrate by using a vacuum pump. Thereafter, ALP (alkaline phosphatase) staining was performed, and the adhesion status of the cells on the upper surface and the recessed portions of the substrate was observed with a digital microscope.

As a result, as shown in Table 1, it was confirmed that 50% of the seeded cells were adhered to inside the wells of the carrier. The pressure loss was too small to perform suctioning uniformly.

### Comparative Example 6

200 g of alumina powder (AS-50, manufactured by Showa Denko K.K.) having an average particle diameter of 9 μm as a raw material, 1 g of polyacrylic acid ammonium (ARON A-30SL, manufactured by Toagosei Co., Ltd.) and 3.5 g of an epoxy resin (DENACOL EX-614B, manufactured by Nagase ChemteX Corporation) as dispersing agents, and 50 g of pure water as a dispersion solvent were stirred and mixed for 15 hours by a ball mill to prepare a raw material slurry. Furthermore, 1 g of 3,3′-diaminodipropylamine (manufactured by Tokyo Chemical Industry Co., Ltd.) as a gelling agent was added, followed by pouring into a mold to obtain an alumina molded product. The molded product was sintered at 1700° C. in air for 2 hours to obtain an alumina ceramic sintered body.

Multiple prescribed wells were formed in the obtained sintered body to provide a first substrate. The thickness of the substrate was set to 11.3 mm, and the thickness from the lowermost portion of the well to the lower surface of the substrate was set to 11 mm. In addition, the diameter of the above well was set to 300 μm and the depth was set to 300 μm.

The average pore diameter of the substrate was 2 μm, and the porosity was 35%. On this substrate was seeded a suspension (5×10⁵ cells/ml) in which 5×10⁵ human iPS cells were suspended in 1 ml of PBS (phosphate buffered saline), followed by sucking at −10 kPa from the bottom of the substrate by using a vacuum pump. Thereafter, ALP (alkaline phosphatase) staining was performed, and the adhesion status of the cells on the upper surface and the recessed portions of the substrate was observed with a digital microscope.

As a result, as shown in Table 1, it was confirmed that 50% of the seeded cells were adhered to inside the wells of the carrier. The substrate was too thick to perform suctioning uniformly.

TABLE 1

| | First substrate | | | Evaluation of carrier | |
|---|---|---|---|---|---|
| | | | Thickness from | Proportion of | |
| | Average | | lowermost | cells adhering to | |
| | pore | | portion of well to | inside wells of | |
| | diameter | Porosity | lower surface of | carrier relative to | |
| | (μm) | (%) | substrate (μm) | seeded cells (%) | Remark |
| Ex. 1 | 0.18 | 40 | 200 | 90 | |
| Ex. 2 | 0.05 | 30 | 50 | 75 | |
| Ex. 3 | 1 | 50 | 500 | 75 | |
| Ex. 4 | 0.18 | 40 | 600 | 60 | |
| Ex. 5 | 1.1 | 40 | 200 | 65 | |
| Ex. 6 | 10 | 30 | 2000 | 60 | |
| Ex. 7 | 2 | 25 | 2000 | 60 | |
| Ex. 8 | 2 | 35 | 10000 | 60 | |
| Com. Ex. 1 | 0.04 | 30 | 200 | 50 | |
| Com. Ex. 2 | 0.09 | 20 | 200 | 50 | |
| Com. Ex. 3 | 0.22 | 55 | 200 | — | Substrate was |
| | | | | | cracked |
| Com. Ex. 4 | 0.18 | 40 | 45 | — | Substrate was |
| | | | | | cracked |
| Com. Ex. 5 | 11 | 40 | 2000 | 50 | Pressure loss was |
| | | | | | too small to |
| | | | | | perform suctioning |
| | | | | | uniformly |
| Com. Ex. 6 | 2 | 35 | 11000 | 50 | Substrate was too |
| | | | | | thick to perform |
| | | | | | suctioning |
| | | | | | uniformly |

As can be seen from Table 1, in a case of providing a first substrate having a plurality of wells on the upper surface and made of a porous body, the porous body having pores having an average pore diameter of 0.05 μm or more and 10 μm or less and having a porosity of 25% or more and 50% or less, in which the first substrate has the thickness from the lowermost portion of the well to the lower surface of the substrate is 50 μm or more and 10 mm or less, it was confirmed that the proportion of cells adhering to inside the wells was as high as 60% to 90%, and the cells can be uniformly collected to the lowermost portion of the well.

### <Preparation of Second Substrate and Cell Culture Carrier, and Evaluation of Cell Culture Carrier>

### Example 9

200 g of alumina powder (AKP-20C, manufactured by Sumitomo Chemical Co., Ltd.) as a raw material powder, 50 g of ion-exchanged water as a dispersion solvent, and 7 g of an epoxy resin (DENACOL EX 614 B, manufactured by Nagase ChemteX Corporation) as an organic substance having crosslinking polymerizability were used and mixed in a ball mill for 5 hours to prepare a slurry. To the slurry was added 4 g of triethanolamine lauryl sulfate (EMAL TD, manufactured by Kao Corporation; anionic surfactant) as a foaming agent, followed by foaming up to reaching 350 cm³ by mechanical stirring to prepare a foamy slurry. 2 g of 3,3′-diaminodipropylamine (manufactured by Tokyo Chemical Industry Co., Ltd.) as a crosslinking agent was added thereto, and the mixture was sufficiently stirred, introduced into a mold, left to stand, and removed from the mold at the time of losing fluidity and exhibiting strength to the extent that handling was possible by crosslinking polymerization. After removed from the mold, the resultant was sufficiently dried by using a humidifying dryer and a dryer, followed by sintering at 1200° C.

The average pore diameter of the communication hole of the alumina sintered body obtained in this manner was 80 μm, and the average pore diameter of the communication portion was 20 μm. In addition, the average pore diameter of the skeleton portion was 0.15 μm. The first substrate (Comparative Example 4) described above was stacked on the upper surface of the second substrate to prepare a cell culture carrier. Thereafter, a suspension (5×10⁵ cells/ml) in which 5×10⁵ human iPS cells were suspended in 1 ml of PBS (phosphate buffered saline) was seeded, followed by sucking at −99 kPa from the bottom of the substrate by using a vacuum pump. Thereafter, ALP (alkaline phosphatase) staining was performed, and the adhesion status of the cells on the upper surface and the recessed portions of the substrate was observed with a digital microscope. As a result, as shown in Table 2, it was confirmed that 90% of the seeded cells were adhered to inside the wells of the carrier.

### Comparative Example 7

200 g of alumina powder (AKP-20C, manufactured by Sumitomo Chemical Co., Ltd.) as a raw material powder, 50 g of ion-exchanged water as a dispersion solvent, and 7 g of an epoxy resin (DENACOL EX 614 B, manufactured by Nagase ChemteX Corporation) as an organic substance having crosslinking polymerizability were used and mixed in a ball mill for 5 hours to prepare a slurry. To the slurry was added 2 g of triethanolamine lauryl sulfate (EMAL TD, manufactured by Kao Corporation; anionic surfactant) as a foaming agent, followed by foaming up to reaching 200 cm³ by mechanical stirring to prepare a foamy slurry. 2 g of 3,3′-diaminodipropylamine (manufactured by Tokyo Chemical Industry Co., Ltd.) as a crosslinking agent was added thereto, and the mixture was sufficiently stirred, introduced into a mold, allowed to stand, and removed from the mold at the time of losing fluidity and exhibiting strength to the extent that handling was possible by crosslinking polymerization. After removed from the mold, the resultant was sufficiently dried by using a humidifying dryer and a dryer, followed by sintering at 1200° C.

The average pore diameter of the communication hole of the alumina sintered body obtained in this manner was 75 μm, and the average pore diameter of the communication portion was 5 μm. In addition, the average pore diameter of the skeleton portion was 0.15 μm.

The first substrate (Comparative Example 4) described above was stacked on the upper surface of the second substrate to prepare a cell culture rather. Thereafter, a suspension (5×10⁵ cells/mi) in which 5×10⁵ human iPS cells were suspended in 1 ml of PBS (phosphate buffered saline) was seeded, followed by sucking at −99 kPa from the bottom of the substrate by using a vacuum pump. Thereafter, ALP (alkaline phosphatase) staining was performed, and the adhesion status of the cells on the upper surface and the recessed portions of the substrate was observed with a digital microscope.

As a result, as shown in Table 2, it was confirmed that 60% of the seeded cells were adhered to inside the wells of the carrier. However, there was variation in the number of cells adhering to inside wells. The result is shown in Table 2.

The second substrate having the communication hole (pore portion), the communication portion and the skeleton portion as shown in Table 2 was prepared in the same process as in Example 9 except that the pore diameter of the communication portion (pore portion) and the pore diameter of the communication hole are changed by varying the amount of the surfactant, the stirring time and the stirring rate. The first substrate (Comparative Example 4) was stacked on the upper surface of the second substrate to prepare cell culture carriers (Examples 10 to 14, Comparative Examples 8 to 12).

Thereafter, a suspension (5×10⁵ cells/ml) in which 5×10⁵ human iPS cells were suspended in 1 ml of PBS (phosphate buffered saline) was seeded, followed by sucking at −99 kPa from the bottom of the substrate by using a vacuum pump. Thereafter, ALP (alkaline phosphatase) staining was performed, and the adhesion status of the cells on the upper surface and the recessed portions of the substrate was observed with a digital microscope. The result is shown in Table 2.

TABLE 2

| | | | | | Evaluation of carrier | |
|---|---|---|---|---|---|---|
| | Second substrate | | | | Proportion of | |
| | Average pore | Average pore | Average | | cells adhering | |
| | diameter of | diameter of | pore diameter | | to inside wells | |
| | communication | communication | of skeleton | First | of carrier relative | |
| | hole | portion | portion | substrate | to seeded cells (%) | Remark |
| Ex. 9 | 80 | 20 | 0.15 | Com. Ex. 4 | 90 | |
| Ex. 10 | 200 | 60 | 0.15 | Com. Ex. 4 | 75 | |
| Ex. 11 | 100 | 20 | 0.15 | Com. Ex. 4 | 85 | |
| Ex. 12 | 200 | 100 | 0.15 | Com. Ex. 4 | 75 | |
| Ex. 13 | 100 | 60 | 0.05 | Com. Ex. 4 | 90 | |
| Ex. 14 | 100 | 60 | 1.0 | Com. Ex. 4 | 75 | |
| Com. Ex. 7 | 75 | 5 | 0.15 | Com. Ex. 4 | 60 | There was |
| | | | | | | variation to the |
| | | | | | | number of cells |
| | | | | | | adhering to |
| | | | | | | inside wells |
| Com. Ex. 8 | 300 | 60 | 0.15 | Com. Ex. 4 | — | Substrate was |
| | | | | | | cracked |
| Com. Ex. 9 | 100 | 10 | 0.15 | Com. Ex. 4 | 60 | There was |
| | | | | | | variation in the |
| | | | | | | number of cells |
| | | | | | | adhering to |
| | | | | | | inside wells |
| Com. Ex. 10 | 200 | 110 | 0.15 | Com. Ex. 4 | — | Substrate was |
| | | | | | | cracked |
| Com. Ex. 11 | 100 | 60 | 0.01 | — | — | Second substrate |
| | | | | | | could not be |
| | | | | | | sintered (cracked |
| | | | | | | during firing) |
| Com. Ex. 12 | 100 | 60 | 1.5 | — | — | Second substrate |
| | | | | | | could not be |
| | | | | | | sintered (cracked |
| | | | | | | during firing) |

Even a substrate on which cracking (breakage) occurs as in Comparative Example 4, in a case where laminated as the first substrate on the upper surface of the second substrate as shown in Examples 9 to 14, it was confirmed that it is possible to efficiently suck cells into the wells of the first substrate while preventing breakage of the first substrate.

While the present invention has been described in detail and with reference to specific embodiments thereof; it will be apparent to one skilled in the art that various changes and modifications can be made therein without departing from the spirit and scope of the present invention.

The present application is based on a Japanese patent application filed on May 27, 2016 (Application No. 2016-106423) and a Japanese patent application filed on Apr. 7, 2017 (Application No. 2017-076482), the content thereof being incorporated herein by reference.

### INDUSTRIAL APPLICABILITY

The cell culture carrier of the present invention can contribute to the development of culturing techniques for undifferentiated cells such as ES cells, adult stem cells and the like and to the application to the regenerative treatment of living tissue.

### REFERENCE SIGNS LIST

1 Cell culture carrier
2 Substrate (first substrate)
2a Front surface
2b Lower surface (rear surface)
3 Well
3a Lowermost portion of well
10 Cell culture carrier
11 Second substrate
11a Front surface
11b Lower surface (rear surface
21A Communication hole
21B Skeleton portion
21a Pore portion
21b Communication portion
P (Suction) Pressure
P1 Negative pressure at lowermost portion of well
P2 Negative pressure at side wall portion of well
P3 Negative pressure on front surface of first substrate
d Well diameter
t1 Thickness of first substrate
t2 Thickness from lowermost portion of well to lower surface of substrate
t3 Thickness of well
t4 Thickness of second substrate

1. A cell culture carrier, comprising a first substrate having a plurality of wells on an upper surface and comprising a porous body, the porous body having pores with an average pore diameter of 0.05 μm or more and 10 μm or less and having a porosity of 25% or more and 50% or less,
wherein the first substrate has a thickness from a lowermost portion of the well to a lower surface of the substrate of 50 μm or more and 10 mm or less.

2. A cell culture carrier comprising:
a first substrate having a plurality of wells on an upper surface, having a thickness from a lowermost portion of the well to a lower surface of the substrate of more than 0 μm and 45 μm or less, and comprising a porous body, the porous body having pores with an average pore diameter of 0.05 μm or more and 10 μm or less and having a porosity of 25% or more and 50% or less; and
a second substrate having a communication hole with an average pore diameter of 50 μm or more and 200 μm or less and with an average pore diameter of a communication portion of 20 μm or more and 100 μm or less, and having a skeleton portion with an average pore diameter of 0.05 μm or more and 1.0 μm or less,
wherein the first substrate is stacked on an upper surface of the second substrate.

3. The cell culture carrier according to claim 1, wherein the well has a diameter of 10 μm or more and 1000 μm or less and a depth of 10 μm or more and 1000 μm or less.

4. The cell culture carrier according to claim 2, wherein the well has a diameter of 10 μm or more and 1000 μm or less and a depth of 10 μm or more and 1000 μm or less.

5. The cell culture carrier according to claim 1, wherein the first substrate comprises at least one kind of ceramic selected from the group consisting of zirconia, yttria, titania, alumina, alumina-silica, silica, hydroxyapatite, and β-tricalcium phosphate.

6. The cell culture carrier according to claim 2, wherein the first substrate and the second substrate independently comprise at least one kind of ceramic selected from the group consisting of zirconia, yttria, titania, alumina, alumina-silica, silica, hydroxyapatite, and β-tricalcium phosphate.

7. The cell culture module comprising the cell culture carrier according to claim 1, wherein the module comprises a suction portion on a rear surface side or/and a pressurizing portion on a front surface side of the cell culture carrier.

8. The cell culture module comprising the cell culture carrier according to claim 2, wherein the module comprises a suction portion on a rear surface side or/and a pressurizing portion on a front surface side of the cell culture carrier.
